(19) United States
(12) Patent Application Publication
(10) Pub. No.: US 2026/0084011 A1
(43) Pub. Date: Mar. 26, 2026

(54) PERSONAL FITNESS GUIDANCE DEVICE WITH QUANTIFIABLE EXERCISE EFFECTS

(71) Applicant: Leao Wang, Taichung (TW)

(72) Inventor: Leao Wang, Taichung (TW)

(21) Appl. No.: 18/891,460

(22) Filed: Sep. 20, 2024

(51) Int. Cl. A63B 24/00; A61B 5/00; A61B 5/024; G16H 20/30; G16H 40/63

(52) U.S. Cl. CPC A63B 24/0062; A61B 5/024; A61B 5/7445; G16H 20/30; G16H 40/63; A63B 2024/0065

(57) ABSTRACT

A personal fitness guidance device with quantifiable exercise effects, comprising a control and display unit that can be securely placed within the user's line of sight for easy viewing, and a detection transmitter that can be attached to the user's body to continuously monitor their heart rate. The control and display unit includes a receiver to continuously receive real-time heart rate data from the detection transmitter. The invention is characterized by its ability to use built-in software and corresponding operational procedures to quantify the user's current physical condition into a biological age. Based on this biological age, the device calculates the user's optimal exercise heart rate zone and displays it using clear, bright, color-coded lights. This allows the user to easily determine whether their current exercise intensity is within the optimal heart rate zone, enabling real-time adjustments to maintain the optimal heart rate indicated by the light.

### BACKGROUND OF THE INVENTION

### 1. Fields of the Invention

The invention relates to a personal fitness guidance device with quantifiable exercise effects. More specifically, it refers to a device designed to accurately measure an exercise user's true physical condition in real-time, quantifying it into a biological age. This serves as the basis for determining the optimal exercise heart rate zone. The device also allows for measurements to be taken before and after exercise, enabling users to instantly assess their fitness progress. It further updates and adjusts the relevant optimal heart rate parameters for guiding future workouts. Additionally, the invention features simple and easy-to-understand bright, color-coded lights to provide clear and effective fitness guidance to the user.

### 2. Description of the Related Art

Before elaborating on the operational features related to the invention, the inventor would like to briefly explain the concept of the so-called “optimal exercise heart rate zone” (also referred to as the target heart rate zone). This term specifically refers to the percentage range of the maximum heart rate of the human body, which is defined as the “optimal exercise heart rate zone” (generally specified between 60-90%). In other words, when engaging in any physical fitness activity, it is recommended to maintain the heart rate within this target range to achieve optimal and safe exercise results. Of course, it is well known that the speed of the human heart rate and pulse at any given time is strongly correlated with age and also reflects the body's oxygen supply capacity. Therefore, the stability and regular fluctuation of heart rate and pulse indirectly indicate the efficiency of cardiac function, blood oxygen supply, or the overall physical condition of the individual. Using the optimal exercise heart rate zone as a reference and guiding indicator for fitness activities is currently considered the safest and most recommended approach for physical training.

At present, the common formula for calculating this is as follows:

$$220 - Actual Age = Maximum Heart Rate$$

$$Maximum Heart Rate \times 60 - 90 \% = Optimal Exercise Heart Rate Zone$$

According to the aforementioned formula, taking a 40-year-old exercise user as an example, their maximum heart rate would be 180 beats per minute. The optimal exercise heart rate zone for this individual would range between 108 and 162 beats per minute. Furthermore, when the exercise heart rate exceeds 163 beats per minute, or even surpasses 180 beats per minute, this would indicate an over-limit exercise load. In such cases, not only is it discouraged for anyone to engage in prolonged activity at this level, but timely intervention or assistance should also be provided to prevent potential dangers.

To assess the current physical condition of the exercise user, the procedure is based on the aforementioned basic parameters. The user should engage in any form of exercise, ensuring that their heart rate remains within the optimal exercise heart rate zone for at least three minutes (the longer the duration, the more accurate the test results). After this, the user should immediately stop exercising and remain at rest for at least two minutes. During this testing process, the “heart rate at the moment of stopping exercise” is measured, and the “heart rate after two minutes of rest” is subtracted from it to calculate a “heart rate recovery value.” In theory, the larger this value, the better the true physical condition of the body, and conversely, the smaller the value, the poorer the condition.

Consequently, after consistently maintaining a fitness regimen for a period of time, an individual's physical condition will generally improve and strengthen. In simple terms, their capacity for a higher exercise heart rate zone may exceed what is expected for their actual age. Therefore, when this situation occurs, the aforementioned universal formula may no longer be adequate. Instead, the latest physical condition (i.e., replacing the actual age) must be used as the basis for calculation to more accurately reflect the individual's true needs.

Based on the widely applied physical fitness testing methods described above, the invention of Taiwan Patent No. I511068, titled “Planning Method and System for Exercise Targets,” was introduced. From the related explanations in this previous patent, it is clear that the system was developed based on the aforementioned formula, serving as a corresponding fitness planning aid. However, it is regrettable that this system merely relies on devices such as computers or mobile phones, along with measuring instruments, to measure heart rate, collect heart rate data, calculate target heart rates, record and store related information, and later provide exercise suggestions (such as recommended exercise types, frequency, or duration). Beyond these functions, the system only serves as a basic measurement and recording device during each fitness session, offering no additional specialized uses. As a result, in practice, such auxiliary systems that only provide exercise recommendations after the fact can quickly become cumbersome and inconvenient for users, leading them to lose interest in long-term use, thereby greatly diminishing the system's practical value.

Additionally, in the prior art of Taiwan Utility Model Patent No. M594228, titled “Luminous Warning Fitness Bike,” and U.S. Pat. No. 6,553,247, titled “ELECTRODE BELT OF HEART RATE MONITOR,” we also observe the emergence of specific products designed to achieve the expected benefits by utilizing real-time monitoring of the user's exercise heart rate in combination with a display device. However, these conventional products are primarily intended for real-time monitoring and displaying the user's heart rate and pulse condition. Aside from offering additional safety warning functions, they provide no substantial assistance in evaluating the fitness effectiveness or guiding the user towards achieving progressive fitness goals.

Of course, many larger fitness centers today require all members to wear heart rate monitors during exercise. These devices transmit real-time heart rate data through corresponding receivers, which then display each individual's heart rate information in designated personal blocks on several large screens. Additionally, these systems are equipped with warning lights to immediately notify the appropriate personnel if any individual's heart rate data shows abnormalities, allowing safety staff to intervene and provide assistance promptly. This setup greatly enhances overall safety in the facility and can be regarded as an excellent and highly practical venue feature. However, it is unfortunate that these systems, while improving safety, still cannot directly offer fitness performance evaluations or guidance for achieving progressive fitness goals for users. Instead, users must rely on nearby professional trainers for necessary exercise instructions.

### SUMMARY OF THE INVENTION

A first object of the invention is to provide a more real-time, accurate, and user-friendly personal assistance device for exercise users. This device allows users to easily adjust their exercise intensity simply by following the color of the light indicators, thereby achieving optimal fitness results.

According to the invention, a control and display unit is securely placed within the exercise user's line of sight for easy, real-time viewing, and a detection transmitter that can be attached to the user's body to continuously monitor their heart rate. The control and display unit includes a receiver that constantly receives real-time heart rate data from the detection transmitter. The invention is characterized by the ability of the display unit to use built-in software and corresponding operational procedures to quantify the user's current physical condition into a biological age. Based on this biological age, the system calculates the user's optimal exercise heart rate zone, which is then displayed using clear and bright color-coded lights. This allows the user to immediately understand, at a glance, whether their current exercise intensity is within the optimal heart rate zone, enabling real-time adjustments to maintain the appropriate heart rate intensity. By following the light color, the user can achieve the desired fitness guidance and results. Additionally, specific warning lights or sounds can be used to indicate potential dangers, such as heart rate overload, ensuring the highest level of exercise safety.

### BRIEF DESCRIPTION OF THE DRAWINGS

The accomplishment of this and other objects of the invention will become apparent from the following description and its accompanying drawings of which:

FIG. 1 is a perspective view of a preferred embodiment of the invention; and

FIG. 2 is a schematic view of the preferred embodiment of the invention shown in FIG. 1, with the cover removed.

### DETAILED DESCRIPTION OF THE PREFERRED EMBODIMENT

The present invention will now be described in more detail hereinafter with reference to the accompanying drawings that show various embodiments of the invention.

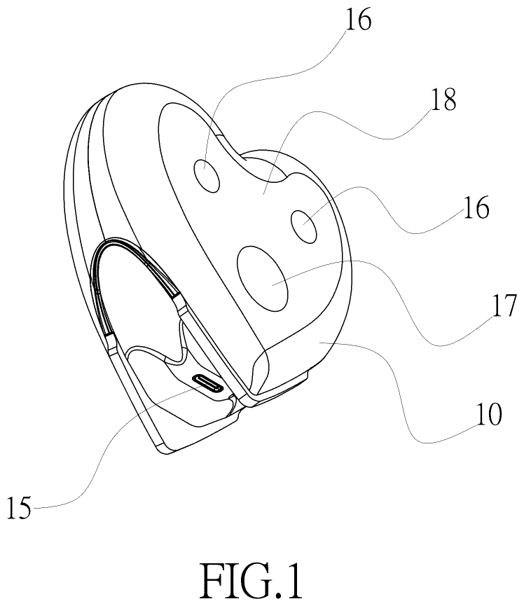

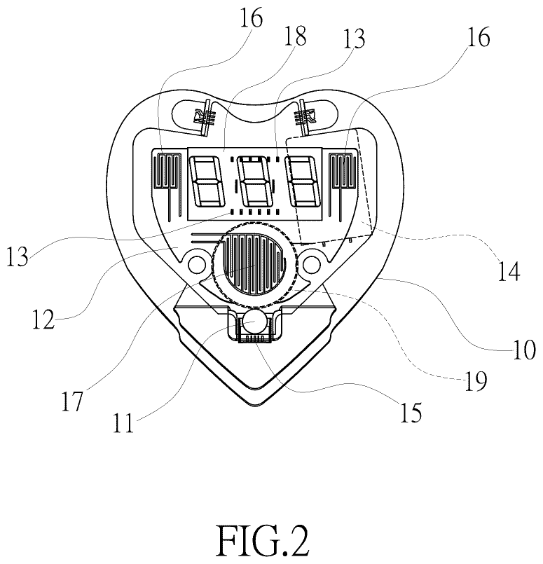

Referring to FIGS. 1 and 2, which illustrate a preferred embodiment of the present invention, it can be seen that the system includes a control and display unit 10 and a heart rate detection transmitter (not shown in the figures).

The control and display unit 10 can be securely placed or magnetically attached at a location visible to the user, allowing for easy viewing at any time. Inside the unit, there is a receiver 11 that continuously receives heart rate data. Additionally, it is equipped with an IC processor 12, containing built-in software for performing relevant calculations, executing commands, and recording information. The unit also includes an LED light assembly 13, a rechargeable battery 14, a charging slot 15, a touch adjustment area 16, a touch activation button 17, and a display screen 18. Furthermore, a magnetic element 19 is provided to allow stable attachment to any metallic surface.

The heart rate detection transmitter (not shown in the figures) that can be securely fastened to the user's body, continuously monitoring and transmitting heart rate data.

Using the aforementioned device, during the initial use, the exercise user must first input their “actual age (ID AGE)” by utilizing the touch adjustment area 16 and touch activation button 17 on the control and display unit 10. At this point, the IC processor 12 will immediately calculate the user's maximum heart rate and optimal exercise heart rate zone based on the program settings. Next, the user performs an initial fitness test using any exercise equipment (such as a treadmill, stationary bike, etc.). This test is based on the “actual age (ID AGE)” as a parameter, where the user exercises continuously within the optimal exercise heart rate zone for more than three minutes, followed by a two-minute rest period, during which the “heart rate recovery value” is measured. The IC processor 12 will then automatically calculate the user's “biological age (BIO AGE)” based on this value. Subsequently, using the “biological age” as a new reference, the system recalculates the user's most accurate maximum heart rate and optimal exercise heart rate zone as follows:

$$220 - Actual Age = Maximium Heart Rate$$

$$Maximium Heart Rate \times 60 - 90 \% = Optimal Exercise Heart Rate Zone$$

This final parameter is then used as the most reliable guide for directing the user's fitness regimen.

Of course, in practical application, the so-called optimal exercise heart rate zone can be freely and reasonably adjusted within an appropriate range, such as 60-80%, 70-80%, or 80-90%, etc. It does not necessarily need to be confined to a specific, fixed value. Additionally, there are other formulas available for estimating the maximum heart rate. These basic formulas can be freely adopted and are not required to follow any particular method exclusively, as clarified here to avoid any misunderstandings.

Moreover, the detection transmitter is, in fact, a commonly known and widely used general-purpose device. Various similar products designed to measure heart rate, whether they are attached to the user's chest, worn on the arm, or the wrist, can seamlessly collaborate with the control and display unit 10 of this invention via the shared principles of the Bluetooth communication protocol without any obstruction. Additionally, the charging slot 15 also functions as a data connection port, allowing the connection to external devices (such as computers or mobile phones) via a corresponding transmission cable (not shown in the figures) to transmit relevant information. However, these features pertain to existing technology and are not the primary focus of this invention, and thus will not be elaborated upon further. This is provided for clarity.

The calculation of the aforementioned “biological age (BIO AGE)” is based on the quantification of the “heart rate recovery value.” The principle behind this is that for any individual with normal cardiac function, their heart rate will accelerate during exercise and gradually decrease after ceasing the activity. Naturally, for the vast majority of people, under the same objective conditions, the heart rate typically decreases by an average number during the transition from an active state to a resting state within a given time frame. For example, if a heart rate drop of 36 beats is taken as the “average benchmark,” then a measured drop greater than this benchmark indicates a better heart rate recovery, while a lower drop suggests poorer recovery. Thus, this invention uses the number of heartbeats decreased as the basis for calculating the “biological age.” Simply put, if the measured heartbeat drop exceeds the benchmark, the “biological age” will be lower than the “actual age (ID AGE).” Conversely, if the drop is less than the benchmark, the “biological age” will be higher. The calculated “biological age” is then immediately displayed on the display screen 18. Regardless of whether the measured “biological age” is higher or lower, the exercise user should receive fitness guidance that aligns with their true physical condition, ensuring that their fitness needs are accurately met.

Assume that a heart rate drop of 36 beats is used as the average benchmark:
A heart rate drop of 37 beats=Actual age−0.1 years (and so on for greater drops)
A heart rate drop of 36 beats=Actual age
A heart rate drop of 35 beats=Actual age+0.1 years (and so on for smaller drops)

The above-mentioned calculation principle not only allows for precise monitoring of each user's true physical condition but also motivates users by clearly presenting accurate data. As a result, it encourages users to pursue continuous improvement in their fitness levels.

After setting the maximum heart rate and optimal exercise heart rate zone based on the “biological age,” the user can perform their fitness routine with the control and display unit 10, following the color guidance shown by the LED light assembly 13. The guidance method uses different colors as progressive indicators. In simple terms, when the user's heart rate has not yet reached the optimal exercise heart rate zone, certain colors (such as cool tones) will indicate insufficient exercise intensity. As the heart rate approaches the optimal zone, the color will gradually change to another tone (such as warm tones) to indicate that the user has entered the optimal heart rate zone. If the heart rate exceeds the target range, a warning color (such as red) will indicate an overloaded heart rate condition. For the user, simply following the color guide allows them to easily maintain their heart rate within the optimal zone by adjusting exercise intensity accordingly. This ensures that they can easily know when to increase or decrease the intensity, helping them consistently stay in the optimal heart rate zone and achieve the best fitness results with ease.

The flashing frequency of the LED light set 13 is synchronized with the heartbeat of the user, meaning that the LED lights gradually brighten and dim in sync with the heartbeat rate, creating a visual effect that gives the observer a genuine feeling of seeing the heart pulsing in unison.

The operational steps of the invention start by using the magnetic element 19 of the control and display unit 10 to securely adhere it within easy sight, such as on the front panel of a treadmill. Then, by entering the actual age through the touch adjustment area 16 and the touch activation button 17, the actual biological age and the optimal heart rate zone for exercise are obtained using the previously described measurement method. Subsequently, the control and display unit 10 will operate the intended fitness guidance function in accordance with these parameters, aided by the detection transmitter. During exercise, users only need to monitor the color of the LED light assembly 13 displayed on the control and display unit 10, striving to maintain their exercise intensity to keep their heartbeat within the optimal heart rate zone. After completing the exercise, users can update their biological age using the same measurement steps and record it. The control and display unit 10 will then recalibrate the optimal heart rate zone based on this updated biological age, guiding the user progressively towards better physical fitness.

Further elaborating, biological age (BIO AGE) can change continuously with the consistency and intensity of fitness training, which contrasts significantly with actual age (ID AGE), which increases by one year annually. This implies that with every exercise session, as the biological age adjusts upwards or downwards, the optimal heart rate zone for exercise also shifts. Thus, calculating the maximum heart rate and the optimal exercise heart rate zone based on biological age is the most accurate and relevant fitness parameter that aligns with an individual's current physical condition. Additionally, this invention generates a new LED color arrangement with each new biological age, offering a clear and lifelike representation of the heartbeat through varying colors. This simple and explicit guidance allows exercise users to focus solely on maintaining their activity within the desired heart rate color zone, eliminating the need to worry about any other trivialities, thereby highlighting its extraordinary practical value.

After each workout session, the user can re-measure and record their biological age to assess whether their physical fitness has improved. This updated biological age will serve as the basis for guiding the next workout session. Notably, the biological age is displayed on the display screen 18 in a precise format, using integers with decimal points (e.g., 39.8 years or 40.2 years). This detailed numerical display creates a significant psychological impact, either motivating or alerting the user, encouraging them to follow the guidance and continue using the system long-term.

The aforementioned formulas for calculating maximum heart rate, optimal exercise heart rate zones, the conversion of heart rate into biological age, and the use of color indicators are provided as examples to assist in illustrating the features of this invention. In practical application, these elements can be selectively adopted or adjusted, and do not limit the scope of the features claimed by this invention.

In summary, the object in accordance with the invention, prior to the application, has not been observed in any similar products available on the market or in related literature. It represents a highly innovative creation based on the natural laws of physics, and it is believed to meet the requirements for the patent application. Therefore, the application is submitted to seek protection under the relevant patent regulations.

What is claimed is:

1. A personal fitness guidance device with quantifiable exercise effects, comprising:
a control and display unit, having a receiver for continuously receiving heart rate data, an IC processor with built-in software for performing relevant calculations, executing commands, and recording information, an LED light assembly, a rechargeable battery, a charging slot, a touch adjustment area, a touch activation button, a display screen, and a magnetic element; and
a heart rate detection transmitter;
wherein the device allows the user to first input their “actual age (ID AGE)” into the control and display unit 10, enabling the IC processor to calculate the user's maximum heart rate and optimal exercise heart rate zone based on programmed settings, then, through a corresponding fitness test procedure, measures the required “heart rate recovery value”, after which the IC processor uses this real physical parameter to calculate the “biological age (BIO AGE)” and recalculates the most accurate maximum heart rate and optimal exercise heart rate zone for guiding the user through the expected fitness routine, and after completing the current fitness session, a new fitness test can be conducted following the same measurement process to obtain and record the updated “biological age” as the reference for guiding the next exercise session.

2. The personal fitness guidance device with quantifiable exercise effects in claim 1, wherein the biological age is displayed on the display screen as an integer with a decimal point.

3. The personal fitness guidance device with quantifiable exercise effects in claim 1, wherein the LED light assembly displays different colored lights to indicate the current exercise intensity, and the flashing frequency gradually changes in synchronization with the user's heart rate.

4. The personal fitness guidance device with quantifiable exercise effects in claim 1, wherein specific warning lights or accompanying warning sounds are provided to indicate potential dangers, such as heart rate overload.
